(19) United States
(12) Patent Application Publication
(10) Pub. No.: US 2017/0112605 A1
(43) Pub. Date: Apr. 27, 2017

(54) PORTABLE ARTIFICIAL RESPIRATOR FOR PET

(71) Applicant: Young-soo ROH, Seoul (KR)

(72) Inventor: Young-soo ROH, Seoul (KR)

(21) Appl. No.: 15/315,703

(22) PCT Filed: Sep. 22, 2015
(86) PCT No.: PCT/KR2015/009960

§ 371 Date: Dec. 1, 2016

(30) Foreign Application Priority Data
Mar. 20, 2015 (KR) 10-2015-0038641

(51) Int. Cl. A61D 7/04; A61M 16/08; A61M 16/20; A61M 16/06; A61M 16/00

(52) U.S. Cl. CPC A61D 7/04; A61M 16/06; A61M 16/0057; A61M 16/208; A61M 16/0875; A61M 16/0048; A61M 2250/00; A61M 2202/0208

(57) ABSTRACT

A portable artificial respirator for a pet comprises: a mask part configured to cover the mouth and nose of the pet and having a mouth opening and a nose opening formed in the front end portion thereof; and an air injection part detachably coupled to the mask part, wherein the air injection part communicates with the mask part while at least a part thereof is inserted into the mouth opening of the mask part when the air injection part is coupled to the mask part. The portable artificial respirator enables a person to safely carry out artificial respiration without bringing his/her mouth into direct contact with a pet's mouth to prevent the pet who recovers consciousness from attacking the person who is providing the artificial respiration and can be assembled, disassembled, and carried in an easy and simple manner.

### TECHNICAL FIELD

The present invention relates to a portable artificial respirator for a pet, and more particularly, to a portable artificial respirator for a pet capable of allowing a cardiopulmonary function of a pet to be revived in an emergency situation of a pet such as a pet dog.

### BACKGROUND ART

Recently, as a population keeping pets such as a pet dog grows, pet-related industries have been developed.

Meanwhile, in a case of foreign countries, when an emergency occurs in which the pet loses consciousness, there are reported cases in which a fire fighter or an owner of the pet has provided the pet with artificial respiration to revive a cardiopulmonary function.

An example of such an artificial respirator for a pet is disclosed in Korean Patent Publication No. 10-2015-0021248 (published on Mar. 2, 2015, hereinafter referred to as “Patent Document 1”).

However, the artificial respirator for a pet of the prior art is not widely distributed because of its complicated structure and high costs. Further, there is a problem that it cannot be easily carried around with the pet due to a weight and a volume of an oxygen tank connected and provided therein.

In addition, there is a problem that a pet who recovers consciousness by artificial respiration may attack a person who is providing the artificial respiration.

### DISCLOSURE

### Technical Problem

Accordingly, it is an object of the present invention to provide a portable artificial respirator for a pet, that allows a person to safely carry out artificial respiration without bringing his/her mouth into direct contact with a mouth of the pet, so as to prevent the pet who recovers consciousness from attacking the person who is providing the artificial respiration.

In addition, another object of the present invention is to provide a portable artificial respirator for a pet, which can effectively inject air into a mouth of the pet only by a breathing action of the person.

Further, another object of the present invention is to provide a portable artificial respirator for a pet, which can be manufactured in a simple structure with low costs and be easily used, while allowing a user to easily disassemble and assemble, and carry the same.

### Technical Solution

In order to achieve the above-described objects, there is provided a portable artificial respirator for a pet, including: a mask part which is configured to cover around a mouth and a nose of the pet and has a mouth opening and a nose opening which are formed in a front end portion thereof; and an air injection part which is detachably coupled to the mask part and is configured to communicate with the mask part while at least a part thereof is inserted into the mask part through the mouth opening with being coupled to the mask part.

The air injection part may include: an outer channel provided at one side thereof so as to be located at an outside of the mask part with being coupled to the mask part; and an inner channel provided at the other side thereof so as to be located at an inside of the mask part and be covered by the mask part with being coupled to the mask part, so that a tip thereof is inserted into the mouth of the pet.

The air injection part may further include: a nose channel provided at a middle portion thereof so as to communicate with the nose opening with being coupled to the mask part.

The mask part may include an exhaust valve provided at one side thereof so as to open an exhaust port formed at the one side toward an outside, when air flowing from the mask part to the air injection part occurs.

At least one of the mask part and the air injection part may include a connection ring which is connected to a lead means of the pet.

The outer channel may further include a slide type whistle mounted on an outside thereof.

The air injection part may be coupled with a hose connected to an oxygen tank.

### Advantageous Effects

In accordance with the portable artificial respirator for a pet according to the present invention, it is possible to allow a person to safely carry out artificial respiration without bringing his/her mouth into direct contact with the mouth of the pet, so as to prevent the pet who recovers consciousness from attacking the person who is providing the artificial respiration.

In addition, in accordance with the portable artificial respirator for a pet according to the present invention, it is possible to effectively inject air into a mouth of the pet only by a breathing action of the person.

Further, in accordance with the portable artificial respirator for a pet according to the present invention, it is possible to manufacture the artificial respirator in a simple structure with low costs and easily use the same, as well as allow a user to easily disassemble and assemble, and carry the same.

### DESCRIPTION OF DRAWINGS

FIG. 1 is an exploded perspective view of a portable artificial respirator for a pet according to a first embodiment of the present invention;

FIG. 2 is an assembled perspective of the portable artificial respirator for a pet of FIG. 1;

FIG. 3 is a side cross-sectional view of the portable artificial respirator for a pet of FIG. 2,

FIGS. 4A and 4B are cross-sectional views illustrating air flow and an operation state of an exhaust valve in a portable artificial respirator for a pet according to a second embodiment of the present invention,

FIG. 5 is a side view illustrating an inside of the portable artificial respirator for a pet of FIGS. 4A and 4B when viewed from a rear to a front of a mask part,

FIG. 6 is a schematic view illustrating a use state of the portable artificial respirator for a pet according to the present invention,

FIG. 7 is a perspective view of a portable artificial respirator for a pet according to a third embodiment of the present invention,

FIG. 8 is a perspective view illustrating a state in which a whistle is coupled to a portable artificial respirator for a pet according to a fourth embodiment of the present invention.

### BEST MODE

Hereinafter, a portable artificial respirator for a pet according to the present invention will be described in detail with reference to the accompanying drawings.

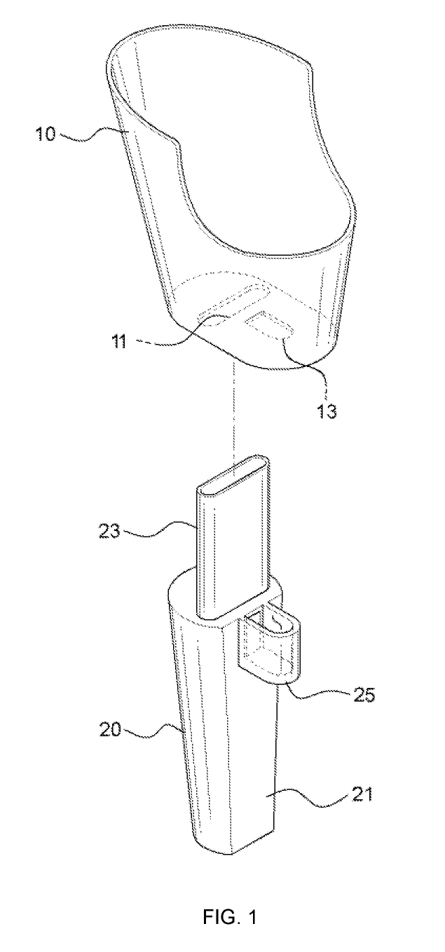

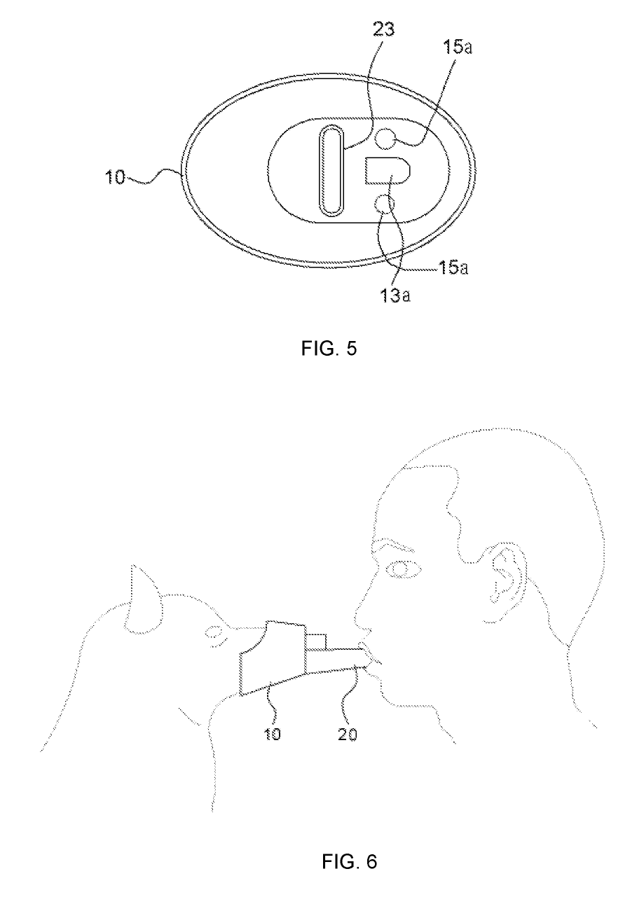

As illustrated in FIGS. 1 and 6, the portable artificial respirator for a pet according to the present invention includes: a mask part 10 which is configured to cover around a mouth and a nose of the pet and has a mouth opening 11 and a nose opening 13 which are formed in a front end portion thereof; and an air injection part 20 which is detachably coupled to the mask part 10 and is configured to communicate with the mask part 10 while at least a part thereof is inserted into the mask part through the mouth opening 11 with being coupled to the mask part 10.

Thereby, it is possible to allow a person to safely carry out artificial respiration without bringing his/her mouth into direct contact with the mouth of the pet, so as to prevent the pet who recovers consciousness from attacking the person who is providing the artificial respiration.

Meanwhile, it is necessary for the mask part 10 to sufficiently cover around the mouth and nose of the pet, thereby it is preferable that the mask part 10 is made of a stretchable material so as to adjust a size thereof according to a size of the pet. Particularly, it is preferable that an edge of a rear end portion contacting with the skin of the pet in the mask part 10 is provided with a curved contour line so as to give a soft feeling, and is made of an elastic material having a higher elasticity than the material of the other portions thereof.

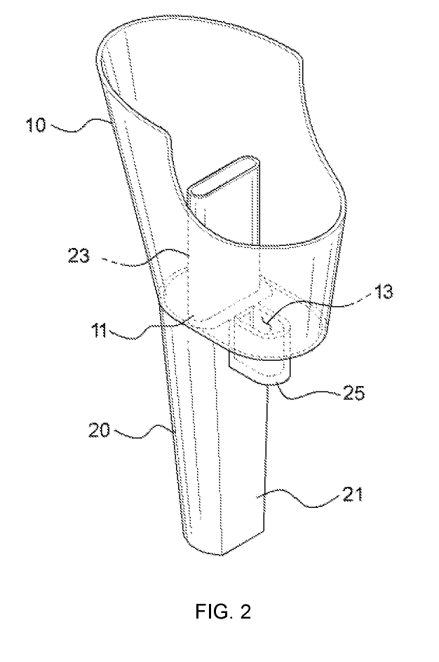

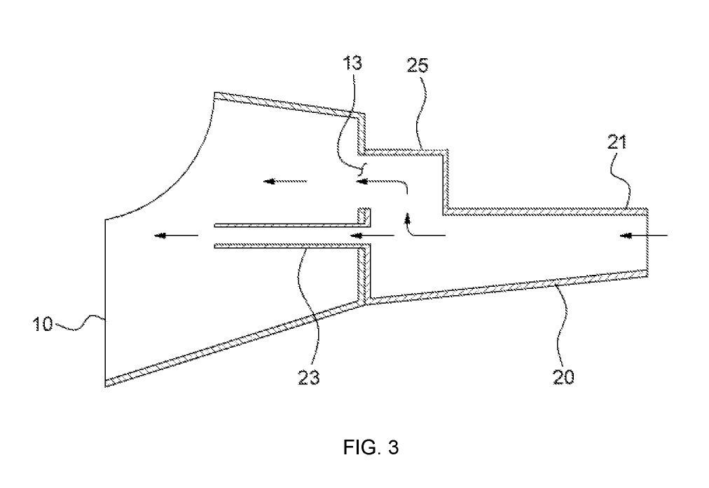

As illustrated in FIGS. 1 to 3, the air injection part 20 includes: an outer channel 21 provided at one side thereof so as to be located at an outside of the mask part 10 with being coupled to the mask part 10; an inner channel 23 provided at the other side thereof so as to be located at an inside of the mask part 10 and be covered by the mask part 10 with being coupled to the mask part 10, so that a tip thereof is inserted into the mouth of the pet; and a nose channel 25 provided at a middle portion thereof so as to communicate with the nose opening 13 with being coupled to the mask part 10.

Accordingly, when the pet is in an emergency situation, the mouth and nose of the pet are covered by the mask part 10, and then the air injection part 20 is coupled to the mask part 10. In this state, when carrying out the artificial respiration through the tip of the outer channel 21, air is entered into the mouth of the pet through the inner channel 23 from the outer channel 21, while air is also entered into the nose of the pet through the nose channel 25 from the outer channel 21 so as to be introduced into a lung. Therefore, air can be effectively supplied to the pet only by the breathing action of the person, and thereby the cardiopulmonary function of the pet can be revived. Herein, the inner channel 23 may be provided at a portion of the air injection part so as to be selectively coupled to the mouth of the pet.

As an embodiment of the present invention, the case in which the air injection part 20 includes the nose channel 25 has been described, but it is not limited thereto, and as another embodiment, the air injection part 20 may be formed only by the outer channel 21 and the inner channel 23 without the nose channel 25.

Further, in a case of not providing the artificial respiration of the person, the air injection part 20 according to the present invention may be coupled with a hose which is connected to an oxygen tank so as to inject oxygen into the pet.

As illustrated in FIGS. 1 to 3, it is preferable that the mask part 10 according to the present invention is inclined in such a manner that an inner space thereof is gradually decreased toward the front end thereof from the rear end contacting with the face portion of the pet, and the outer channel 21 of the air injection part 20 is inclined in such a manner that an inner space thereof is gradually decreased from one end contacting with the front end of the mask part 10 to the other end inserted into the mouth of the person who is providing the artificial respiration when the inner channel 23 is coupled to the mask part 10 through the mouth opening 11.

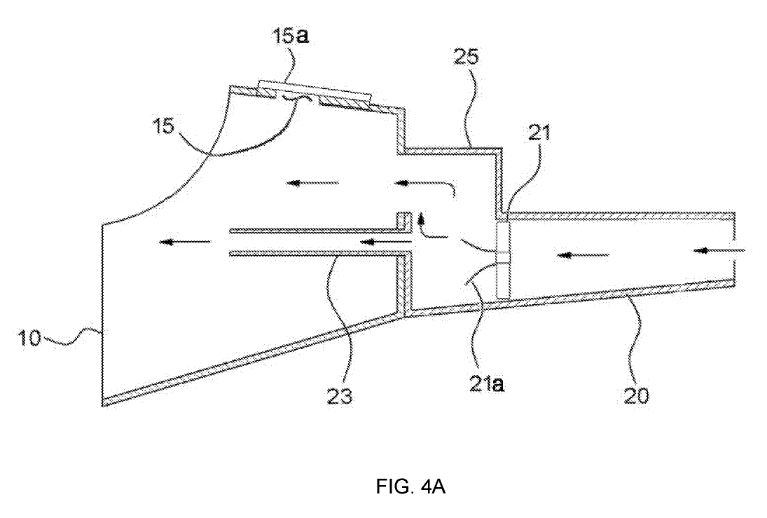

Meanwhile, FIG. 4A is a view illustrating a state in which a person breathes and the breathing is injected into the mouth of the pet when artificial respiration is carried out using a portable artificial respirator for a pet according to another embodiment of the present invention, and FIG. 4B is a view illustrating a state in which the flow of air is prevented from flowing back to the person when the animal is breathing.

As another embodiment of the present invention, as illustrated in FIGS. 4A and 4B, the mask part 10 includes an exhaust valve 15a provided at one side thereof so as to open an exhaust port 15 formed at the one side of the mask part 10 toward an outside, when air flowing from the mask part 10 to the air injection part 20 occurs.

Thereby, when carrying out the artificial respiration using the portable artificial respirator for a pet according to the present invention, the breath exhaled by the pet is bypassed through the opening of the exhaust valve 15a to be discharged to the outside, so that it is possible to safely protect the person who is providing the artificial respiration from the risk of infection with bacteria and viruses that the pet may have.

Meanwhile, the mask part 10 may further include a nose valve provided at an outside thereof so as to open the nose opening 13 when the respiration of the person is injected into the nose channel 25 from the outer channel 21 and close the nose opening 13 when the respiration is reversely flowed. As a result, it is possible to prevent the respiration of the pet from being introduced into the mouth of the person by closing the nose valve.

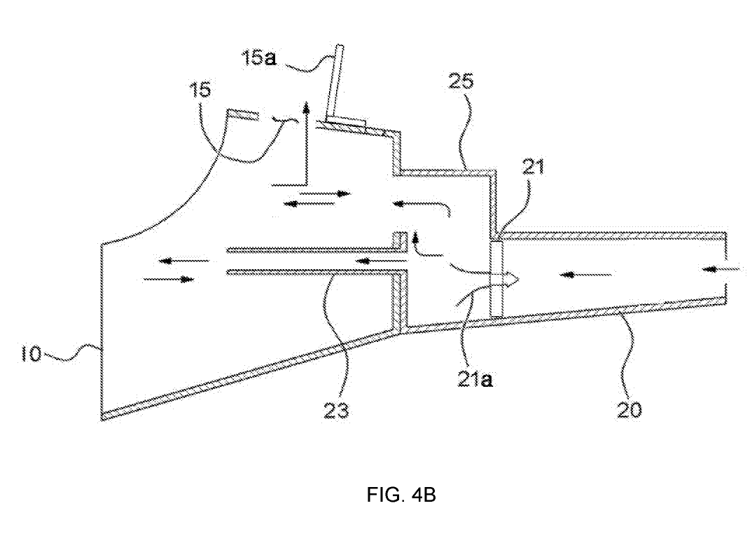

It is preferable that the exhaust port 15 and the exhaust valve 15a according to the present invention are provided at a position near the nose opening 13 in the mask part 10 in consideration of the fact that pets mainly breathe through the nose. Referring to FIGS. 4A and 4B, the configuration, in which the exhaust port 15 and the exhaust valve 15a are arranged at an upper side of a portion where the nose of the pet is located when the pet is wearing the mask part 10, that is an upper portion adjacent to the front end portion of the mask part 10, will be described, but it is not limited thereto, and as illustrated in FIG. 5, these parts may be provided on both sides of the front portion of the mask part 10 with the nose opening 13 interposed therebetween.

Meanwhile, as another embodiment of the present invention, as illustrated in FIGS. 4A and 4B, a mouth valve 21a may be provided between the outer channel 21 and the inner channel 23 so as to open a pathway formed therein when the respiration of the person is injected into the mask part 10 from the outer channel 21 through the inner channel 23 and close the pathway when the respiration is reversely flowed.

Thereby, when carrying out the artificial respiration using the portable artificial respirator for a pet according to the present invention, a mouth valve 21a is opened, while the exhaust valve 15a is opened by the breath exhaled by the pet to be discharged to the outside, and the mouth valve 21a is simultaneously closed, so that it is possible to safely protect the person who is providing the artificial respiration from the risk of infection with bacteria and viruses that the pet may have.

As one embodiment of the present invention, it is preferable that the exhaust valve 15a and the mouth valve 21a are formed in a membrane made of an elastic material such as rubber or polymer, while one ends thereof are fixed to the mask part and the air injection part and the other ends serve as a free end, respectively.

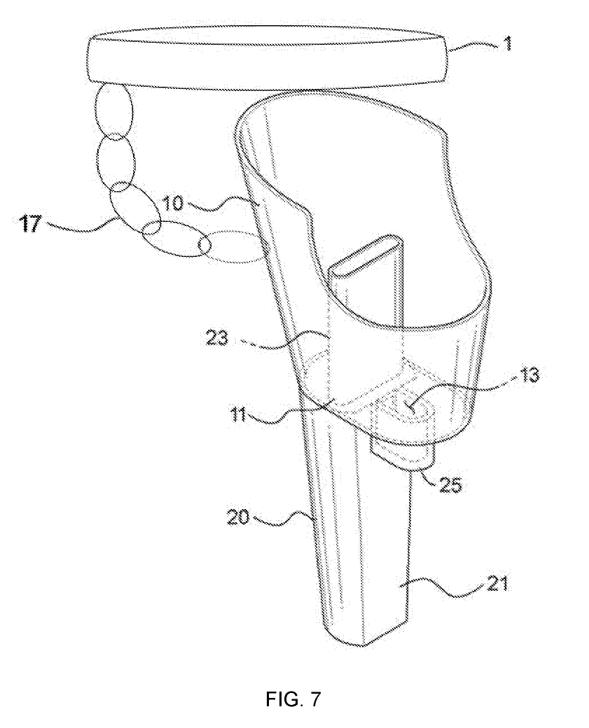

In addition, as another embodiment of the present invention, as illustrated in FIG. 7, at least one of the mask part 10 and the air injection part 20 includes a connection ring 17 which is connected to a lead means 1 of the pet.

Accordingly, a caregiver of the pet may always carry the portable artificial respirator for a pet according to the present invention together with the pet lead means 1 in a convenient manner, and when an emergency situation occurs, it is possible to quickly take emergency measures for the pet using the artificial respirator.

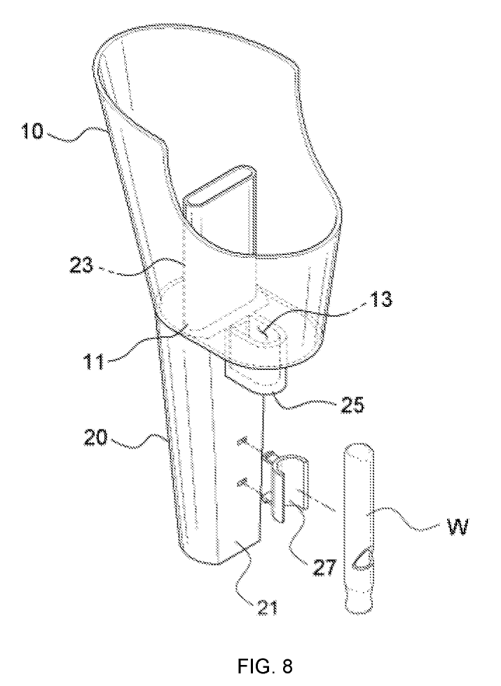

FIG. 8 is a perspective view illustrating a portable artificial respirator for a pet according to a fourth embodiment of the present invention. As illustrated in FIG. 8, the portable artificial respirator for a pet according to the fourth embodiment of the present invention may include an insertion part 27 fixed to the outside of the air injection part 20 in a hook method, and a whistle W mounted in the insertion part 27 in a slide method. Thereby, it is possible to promptly indicate the emergency situation of the pet and request for help using the whistle so as to take a necessary action before it is too late, while it is also possible to train military police dogs using the same. In FIG. 8, the insertion portion 27 is illustrated to be formed on the outer channel 21 side of the air injection part 20, but it is not limited thereto, and the insertion part 27 may be formed on a side face of the air injection part 20 or a side opposite to the outer channel 21.

Thus, according to the present invention, it is possible to provide a portable artificial respirator for a pet that can be easily manufactured in a simple structure with low costs, can be easily disassembled and assembled, and carried.

While the present invention has been described with reference to the preferred embodiments, the present invention is not limited to the above-described specific embodiments, and it will be understood by those skilled in the related art that various modifications and variations may be made therein without departing from the scope of the present invention as defined by the appended claims.

1. A portable artificial respirator for a pet, comprising:
a mask part which is configured to cover around a mouth and a nose of the pet and has a mouth opening and a nose opening which are formed in a front end portion thereof; and
an air injection part which is detachably coupled to the mask part and is configured to communicate with the mask part while at least a part thereof is inserted into the mask part through the mouth opening with being coupled to the mask part.

2. The portable artificial respirator for a pet according to claim 1, wherein the air injection part comprises:
an outer channel provided at one side thereof so as to be located at an outside of the mask part with being coupled to the mask part; and
an inner channel provided at the other side thereof so as to be located at an inside of the mask part and be covered by the mask part with being coupled to the mask part, so that a tip thereof is inserted into the mouth of the pet.

3. The portable artificial respirator for a pet according to claim 2, wherein the air injection part further comprises: a nose channel provided at a middle portion thereof so as to communicate with the nose opening with being coupled to the mask part.

4. The portable artificial respirator for a pet according to claim 3, wherein the mask part includes an exhaust valve provided at one side thereof so as to open an exhaust port formed at the one side toward an outside, when air flowing from the mask part to the air injection part occurs.

5-7. (canceled)

8. The portable artificial respirator for a pet according to claim 1, wherein at least one of the mask part and the air injection part includes a connection ring which is connected to a lead means of the pet.

9. The portable artificial respirator for a pet according to claim 2, wherein at least one of the mask part and the air injection part includes a connection ring which is connected to a lead means of the pet.

10. The portable artificial respirator for a pet according to claim 3, wherein at least one of the mask part and the air injection part includes a connection ring which is connected to a lead means of the pet.

11. The portable artificial respirator for a pet according to claim 4, wherein at least one of the mask part and the air injection part includes a connection ring which is connected to a lead means of the pet.

12. The portable artificial respirator for a pet according to claim 2, wherein the outer channel further includes a slide type whistle mounted on an outside thereof.

13. The portable artificial respirator for a pet according to claim 3, wherein the outer channel further includes a slide type whistle mounted on an outside thereof.

14. The portable artificial respirator for a pet according to claim 4, wherein the outer channel further includes a slide type whistle mounted on an outside thereof.

15. The portable artificial respirator for a pet according to claim 1, wherein the air injection part is coupled with a hose connected to an oxygen tank.

16. The portable artificial respirator for a pet according to claim 2, wherein the air injection part is coupled with a hose connected to an oxygen tank.

17. The portable artificial respirator for a pet according to claim 3, wherein the air injection part is coupled with a hose connected to an oxygen tank.

18. The portable artificial respirator for a pet according to claim 4, wherein the air injection part is coupled with a hose connected to an oxygen tank.
